(19) United States
(12) Patent Application Publication
(10) Pub. No.: US 2015/0064723 A1
(43) Pub. Date: Mar. 5, 2015

(54) DUAL MODE SENSOR

(71) Applicant: Drexel University, Philadelphia, PA (US)

(72) Inventors: Rajakkannu Mutharasan, West Chester, PA (US); Blake N. Johnson, Plainsboro, NJ (US)

(21) Appl. No.: 14/467,493

(22) Filed: Aug. 25, 2014

Related U.S. Application Data
(60) Provisional application No. 61/871,991, filed on Aug. 30, 2013.

(51) Int. Cl. G01N 33/543; G01N 29/02; G01N 27/02

(52) U.S. Cl. CPC G01N 33/54373; G01N 27/02; G01N 29/022; G01N 2291/021; G01N 2291/022; G01N 2291/02425; G01N 2291/02433; G01N 2291/02466; G01N 2291/0427; G01N 2291/018; G01N 2291/014
USPC 435/7.23; 436/501; 435/7.37; 435/7.32

(57) ABSTRACT

A novel dual mode sensor may combine mass-sensing measurements of dynamic-mode cantilevers with electrochemical impedance spectroscopy employed for transduction in sensitive electrochemical biosensors. The integrated design of the sensor may provide simultaneous and continuous measurement of resonant frequency shift and charge transfer resistance of a target analyte bound to a surface of the sensor. Binding of a target analyte to the surface of the sensor may cause charge transfer resistance to increase and the resonant frequency of the sensor to decrease. These simultaneous dynamic modes of the sensor may be utilized to measure an amount of mass of the target analyte accumulated on the surface of the sensor and to reduce and/or eliminate false negative measurement results.

### CROSS REFERENCE TO RELATED APPLICATIONS

This application claims priority to U.S. provisional patent application No. 61/871,991, filed Aug. 30, 2013. U.S. provisional patent application No. 61/871,991 is incorporated herein by reference in its entirety.

### TECHNICAL FIELD

The technical field generally relates to sensors and more specifically relates to sensors comprising multiple sensing modes.

### BACKGROUND

In critical applications such as healthcare, food safety, environmental monitoring, or the like, false negatives regarding biosensing are not tolerated as the consequential damages may be significant.

### SUMMARY

A novel dual mode electrochemical piezoelectric-excited millimeter cantilever sensor may be utilized for simultaneous in-liquid biochemical sensing. The sensor may combine mass-sensing measurements of dynamic-mode cantilevers with electrochemical impedance spectroscopy employed for transduction in sensitive electrochemical biosensors. The integrated design of the sensor may provide simultaneous and continuous measurement of resonant frequency shift and charge transfer resistance of a target analyte bound to a surface of the sensor. Binding of a target analyte to the surface of the sensor may cause charge transfer resistance to increase and the resonant frequency of the sensor to decrease. These simultaneous dynamic modes of the sensor may be utilized to measure an amount of mass of the target analyte accumulated on the surface of the sensor and to reduce and/or eliminate false negative measurement results.

### BRIEF DESCRIPTION OF THE DRAWINGS

The foregoing summary, as well as the following detailed description, may be better understood when read in conjunction with the appended drawings.

FIG. 1 is an illustration of an example piezoelectric cantilever sensor comprising a piezoelectric portion and a non-piezoelectric portion.

FIG. 2 is a cross-sectional view of an example piezoelectric cantilever sensor depicting electrode placement regions.

FIG. 3 is a cross-sectional view of an example piezoelectric cantilever sensor depicting electrode placement regions.

FIG. 4 is another cross-sectional view of an example piezoelectric cantilever sensor depicting other electrode placement regions.

FIG. 5 is an example schematic representation of charge transfer at the working electrode surface of an example sensor.

FIG. 6 is another example illustration of surface binding causing charge transfer resistance increase and resonant frequency decrease in a single sensor with dual transduction.

FIG. 7 depicts modeling measurement of R_{CT} via an ePEMC.

FIG. 8 illustrates schematics and photograph of the ePEMC sensor utilized in the experiments.

FIG. 9 is a plot of imaginary impedance versus real impedance obtained by a fit of data to a modified Randles equivalent circuit model.

FIG. 10 illustrates example resonant mode graphs of phase angle versus frequency.

FIG. 11 is a schematic diagram of an example circuit utilized to depose gold on the surface of the sensor.

FIG. 12 illustrates example plots of ePEMC resonant frequency.

FIG. 13 illustrates example plots of resonant frequency and current.

FIG. 14 illustrates example plots of imaginary impedance.

FIG. 15 illustrates example plots of resonant frequency shift versus time.

FIG. 16 illustrates plots of charge transfer resistance changes versus time.

FIG. 17 illustrates a pictorial comparison of the ePEMC sensor with a commercially available electrochemical-quartz crystal microbalance (EQCM) sensor.

FIG. 18 is a pictorial view of the sensor working electrodes.

FIG. 19 comprises a pictorial illustration of the flow cells used for EQCM sensing and ePEMC sensing and example velocity profiles.

FIG. 20 is a flow diagram of an example process for facilitating dual mode sensing on a single sensor.

FIG. 21 is a block diagram of an example apparatus that may be utilized to effectuate dual mode sensing.

### DETAILED DESCRIPTION OF ILLUSTRATIVE EMBODIMENTS

A dual mode electrochemical piezoelectric-excited millimeter cantilever (ePEMC) sensor may be utilized for simultaneous in-liquid biochemical sensing. The ePEMC may incorporate mass-sensing measurement of dynamic-mode cantilevers along with electrochemical impedance spectroscopy (EIS). EIS may be utilized to measure transduction. Such an integrated design may allow for simultaneous and continuous measurement of resonant frequency shift (Δf) and charge transfer resistance (RCT) as a target analyte binds to a surface of the sensor. In various example embodiments, the surface may comprise any appropriate material or materials with which a target analyte may bind. For example, the surface may comprise a gold coating. The coating may be of any appropriate surface area, such as, for example, 0.5 mm². The resonant frequency shift (Δf) and charge transfer resistance (RCT) may be measured via electromechanical and electrochemical impedance spectroscopy, respectively.

Three experiments were conducted to demonstrate ePEMC properties. The three experiments included: (1) resonant frequency response to electrochemically-deposited metal thin-films, (2) resonant frequency response to adsorption of thiolated ssDNA and model proteins with subsequent EIS sensing, and (3) simultaneous resonant frequency and charge transfer resistance response to model chemisorption of a short-chain thiol molecule, mercaptohexanol. It was observed that adsorption of all model binding analytes caused a decrease in sensor resonant frequency and increase in charge transfer resistance. Comparison of sensor response to binding of protein and thiol molecules showed the two simultaneously transduced signals were proportional and showed similar kinetics.

A biosensor may use electrical, electrochemical, optical, thermal, or electromechanical transduction to convert a molecular binding event into a measureable signal. In critical applications such as healthcare, food safety, and environmental monitoring, false negatives are not tolerated as the consequential damage may be significant. Therefore, a biosensor that provides redundancy and/or dual transduction responses may be useful for verifying a response of one transduction with a second response, and thus, provide a mechanism for reducing or potentially eliminating false negatives.

Electrochemical and resonance-based transduction mechanisms may be utilized for biosensing applications in order to take advantage of their high sensitivity and label-free protocols. For example, detection at femtogram (fg)/mL to picogram (pg)/mL levels may be accomplished with a variety of biological analytes, such as toxins, pathogens, and nucleic acids. Described herein is a robust technique in which binding of target analyte causes two independent transduction responses on the same sensor surface, and both are measured simultaneously.

To achieve the aforementioned simultaneous measurements, an electrochemical approach is integrated with electromechanical transduction in the same device. The measurement of an electrochemical response in biosensing may be facilitated by the selection of an electro-active target or electro-active labeled-reagent for facilitating electrochemical measurement. However, labeling recognition molecules, such as antibodies, may reduce their avidity, and thus, reduce assay sensitivity. On the other hand, label-free electrochemical measurement via redox probes may be advantageous. Therefore, as described herein, label-free electrochemistry is combined with a label-free piezoelectric-excited millimeter-sized cantilever (PEMC) sensor. The PEMC sensor may exhibit high sensitivity at picomolar (fM) to femtomolar (fM) levels. Likewise, label-free electrochemical biosensors may yield comparably sensitive results. Results are described herein of utilizing an electrochemical-PEMC (ePEMC) which allows simultaneous EIS and mass-change measurement capabilities. Observed results of an electrochemical-quartz crystal microbalance (EQCM) and an ePEMC are described herein. Observed distinguishing features of the ePEMC from EQCM include: (1) vibration is transverse for ePEMC vs. lateral for EQCM, (2) sensing area is square millimeters for ePEMC vs. square centimeters for EQCM, (3) mass change sensitivity is fg/Hz for ePEMC vs. ng/Hz for EQCM, and (4) electrodes used for actuation and sensing are different for ePEMC vs. the same for EQCM.

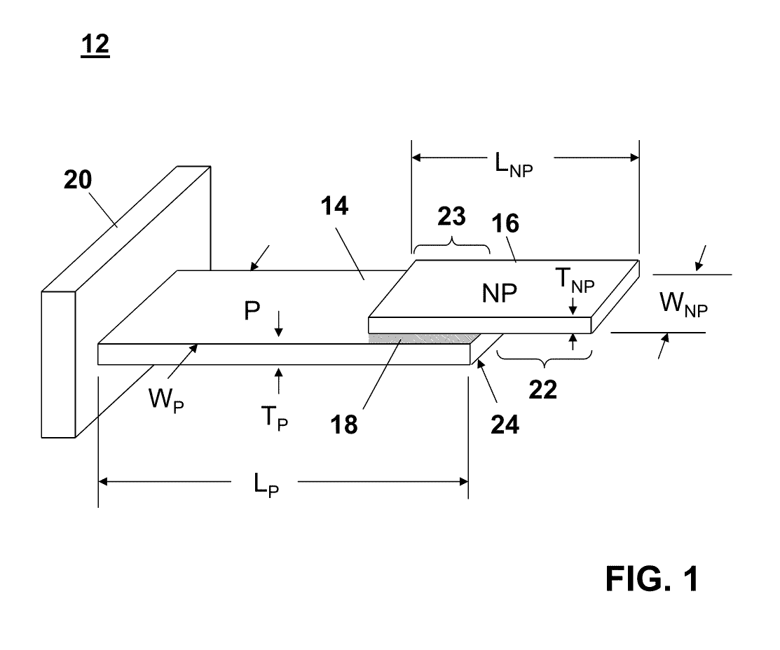

FIG. 1 is an illustration of an example piezoelectric cantilever sensor 12 comprising a piezoelectric portion 14 and a non-piezoelectric portion 16. Piezoelectric portions are labeled with an uppercase letter p (“P”), and non-piezoelectric portions are labeled with the uppercase letters np (“NP”). The piezoelectric cantilever sensor 12 depicts an embodiment of an unanchored, overhang, piezoelectric cantilever sensor. The piezoelectric cantilever sensor 12 is termed “unanchored” because the non-piezoelectric layer 16 is not attached to the base portion 20. The piezoelectric cantilever sensor 12 is termed, “overhang” because the non-piezoelectric layer 16 extends beyond the distal tip 24 of the piezoelectric layer 14 to create an overhanging portion 22 of the non-piezoelectric layer 16. The piezoelectric portion 14 is coupled to the non-piezoelectric portion 16 via adhesive portion 18. The piezoelectric portion 14 and the non-piezoelectric portion overlap at region 23. The adhesive portion 18 is positioned between the overlapping portions of the piezoelectric portion 14 and the non-piezoelectric portion 16. The piezoelectric portion 14 is coupled to a base portion 20.

The piezoelectric portion 14 can comprise any appropriate material such as lead zirconate titanate, lead magnesium niobate-lead titanate solid solutions, strontium lead titanate, quartz silica, piezoelectric ceramic lead zirconate and titanate (PZT), piezoceramic-polymer fiber composites, or the like, for example. The non-piezoelectric portion 16 can comprise any appropriate material such as glass, ceramics, metals, polymers and composites of one or more of ceramics, and polymers, such as silicon dioxide, copper, stainless steel, titanium, or the like, for example.

The piezoelectric cantilever sensor can comprise portions having any appropriate combination of dimensions. Further, physical dimensions can be non-uniform. Thus, the piezoelectric layer and/or the non-piezoelectric layer can be tapered. For example, the length (e.g., L_{P} in FIG. 1) of the piezoelectric portion (e.g., piezoelectric portion 14) can range from about 0.1 to about 10 mm. The length (e.g., L_{NP} in FIG. 1) of the non-piezoelectric portion (e.g., non-piezoelectric portion 16) can range from about 0.1 to about 10 mm. The overlap region (e.g., overlap region 23) can range from about 0.1 to about 10 mm in length. The width (e.g., W_{P} in FIG. 1) of the piezoelectric portion (e.g., piezoelectric portion 14), and the width (e.g., W_{NP} in FIG. 1) of the non-piezoelectric portion (e.g., non-piezoelectric portion 16), can range from about 0.1 mm to about 4.0 mm. The width (e.g., W_{P} in FIG. 1) of the piezoelectric portion can differ from the width (e.g., W_{NP} in FIG. 1) of the non-piezoelectric portion as well. The thickness of the (e.g., T_{P} in FIG. 1) of the piezoelectric portion (e.g., piezoelectric portion 14), and the thickness (e.g., T_{NP} in FIG. 1) of the non-piezoelectric portion (e.g., non-piezoelectric portion 16), can range from about 10 micrometers (10×10⁻⁶ meters) to about 4.0 mm. The thickness (e.g., T_{P} in FIG. 1) of the piezoelectric portion also can differ from the thickness (e.g., T_{NP} in FIG. 1) of the non-piezoelectric portion.

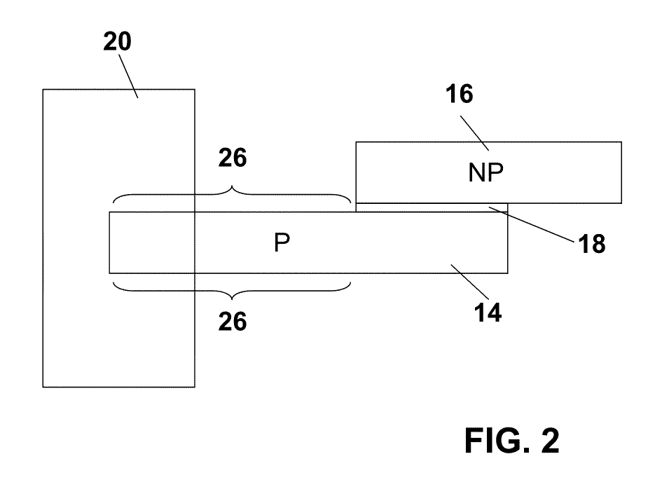

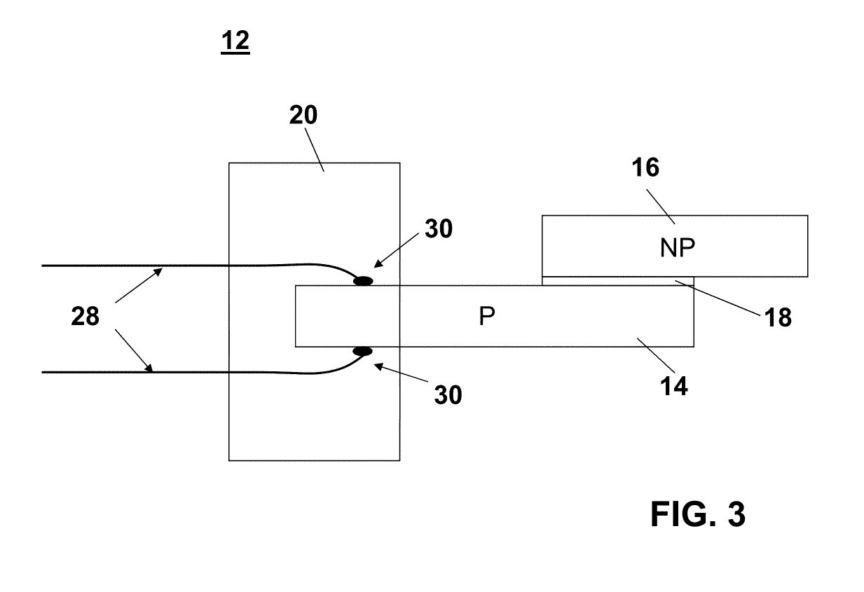

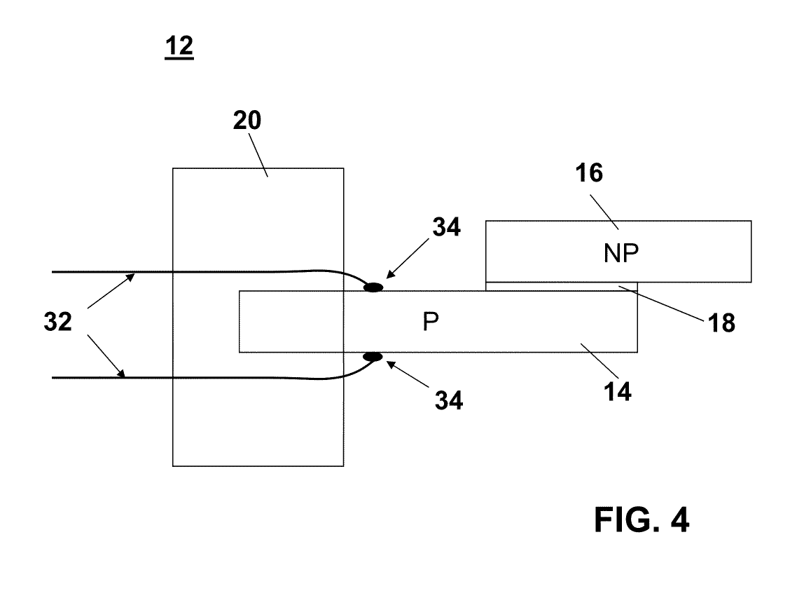

FIG. 2 is a cross-sectional view of the piezoelectric cantilever sensor 12 depicting electrode placement regions 26 for electrodes operationally associated with the piezoelectric portion 14. Electrodes can be placed at any appropriate location on the piezoelectric portion of the piezoelectric cantilever sensor as indicated by brackets 26. For example, as shown in FIG. 3, electrodes 28 can be coupled to the piezoelectric portion 14 within the base portion 20. Or, as depicted in FIG. 4, electrodes 32 can be coupled to the piezoelectric portion 14 at any location not within the base portion 20 and not overlapped by the non-piezoelectric portion 16. Electrodes need not be placed symmetrically about the piezoelectric portion 14. In an example embodiment, one electrode can be coupled to the piezoelectric portion 14 within the base portion 20 and the other electrode can be coupled to the piezoelectric portion 14 not within the base portion 20. Electrodes, or any appropriate means (e.g., inductive means, wireless means), can be utilized to provide an electrical signal to and receive an electrical signal from the piezoelectric portion 14. In an example embodiment, electrodes can be coupled to the piezoelectric portion 14 via a bonding pad or the like (depicted as elements 30 in FIG. 3 and elements 34 in FIG. 4). Example bonding pads can comprise any appropriate material (e.g., gold, silicon oxide) capable of immobilization of a receptor material and/or an absorbent material appropriate for use in chemical sensing or for bio-sensing.

Electrodes may be placed at any appropriate location. In an example embodiment, electrodes may be operatively located near a location of concentrated stress in the piezoelectric layer 14. As described above, the sensitivity of the piezoelectric cantilever sensor is due in part to advantageously directing (concentrating) the stress in the piezoelectric layer 14 and placing electrodes proximate thereto. The configurations of the piezoelectric cantilever sensor described herein (and variants thereof) tend to concentrate oscillation associated stress in the piezoelectric layer 14. At resonance, in some of the configurations of the piezoelectric cantilever sensor, the oscillating cantilever concentrates stress in the piezoelectric layer 14 toward the base portion 20. This may result in an amplified change in the resistive component of the piezoelectric layer 14, and a large shift in resonance frequency at the locations of high stress. Directing this stress to a portion of the piezoelectric layer 14 having a low bending modulus (e.g., more flexible) allows for exploitation of the associated shift in resonance frequency to detect extremely small changes in mass of the piezoelectric cantilever sensor. Thus, in example configurations of the piezoelectric cantilever sensor, the thickness of the piezoelectric layer 14 located near the base portion 20 is thinner than portions of the piezoelectric layer 14 further away from the base portion 20. This may tend to concentrate stress toward the thinner portion of the piezoelectric layer 14. In example configurations, electrodes may be located at or near the locations of the oscillation associated concentrated stress near the base portion of the piezoelectric cantilever sensor. In other example configurations of the piezoelectric cantilever sensor electrodes are positioned proximate the location of concentrated stress in the piezoelectric layer regardless of the proximity of the concentrated stress to a base portion of the piezoelectric cantilever sensor.

The description of piezoelectric cantilever sensors as depicted in FIG. 1 through FIG. 4 and associated text also is applicable to single layer piezoelectric cantilever sensor.

Electrochemical impedance spectroscopy (EIS), also referred to as dielectric spectroscopy or impedance spectroscopy, may be utilized to measure dielectric properties of a medium. Dielectric properties may be measured as a function of frequency. Various properties may be measured, such as, for example impedance and charge transfer resistance (R_{CT}). R_{CT} represents the resistance that a current experiences when cross an electrode/electrolyte interface. EIS may be achieved by applying an electric field to the medium as measured the desired properties.

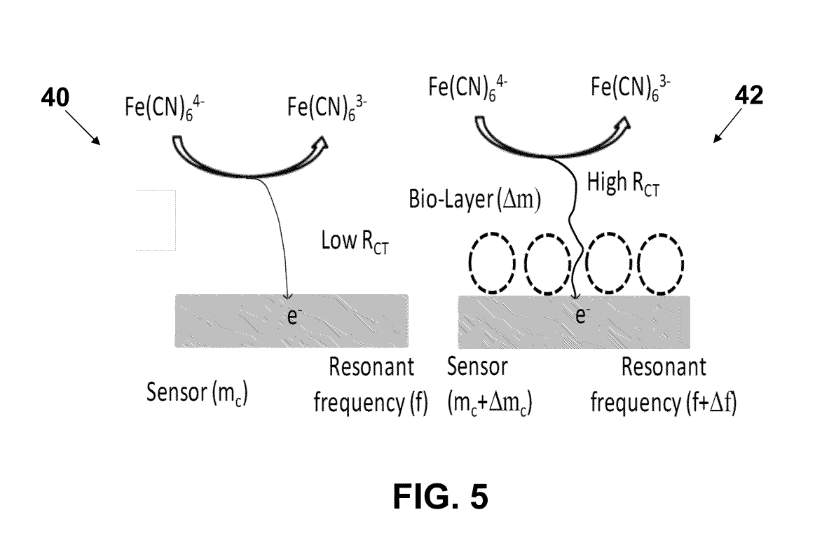

FIG. 5 is an example schematic representation of charge transfer at the working electrode surface of an example sensor. Diagram 40 illustrates transfer across a clean surface. Diagram 42 illustrates charge transfer when the working electrode is populated with adsorbed species (e.g., ssDNA, BSA, and MCH).

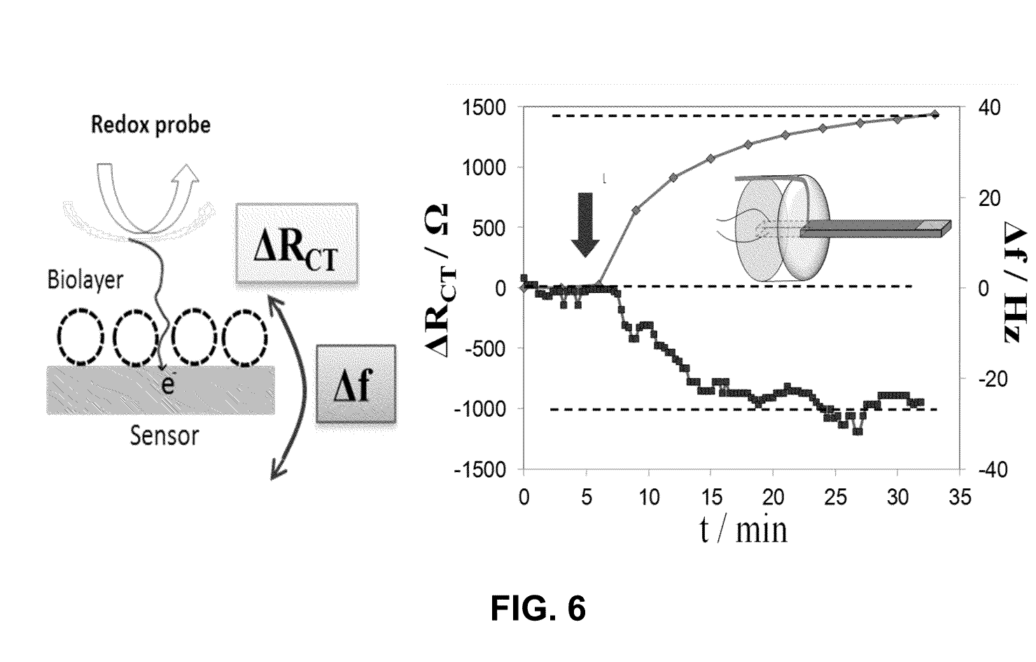

FIG. 6 is another example illustration of surface binding causing charge transfer resistance increase and resonant frequency decrease in a single sensor with dual transduction.

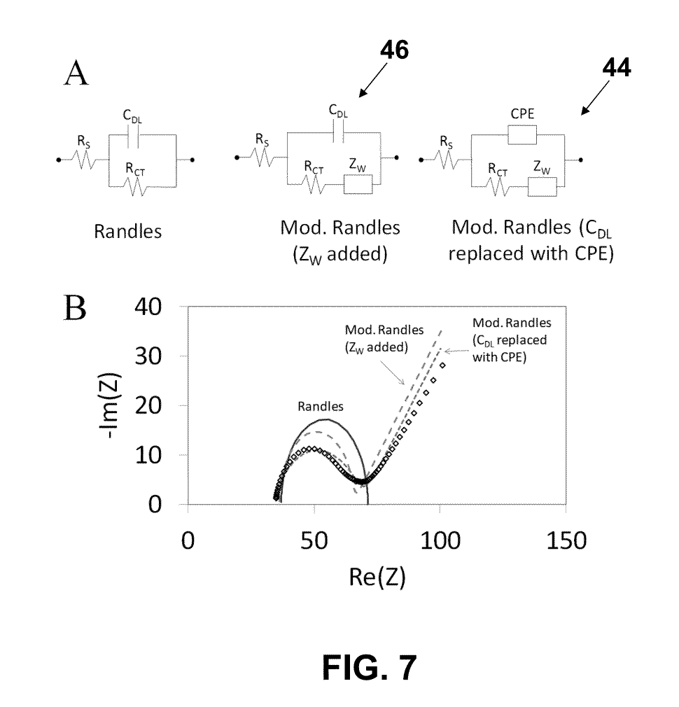

FIG. 7 depicts modeling measurement of R_{CT} via an ePEMC. Diagram A depicts schematics of equivalent circuit models for an ePEMC. Diagram B depicts graphical fits of the equivalent circuit models of diagram A. The graphical data depicted in diagram B may be determined in any appropriate manner. In an example embodiment, the graphical data depicted in diagram B may be determined via the Simplex Method (e.g., Gamry Analyst Software) to ePEMC EIS data. The models depicted in FIG. 7 accurately capture the biosensing parameter R_{CT} (34, 29, and 35Ω, respective to the order shown in FIG. 7). The values compared well with the semicircle distance of 34Ω. Best fit was obtained using the modified Randles circuit 44, which contains a constant phase element (CPE) in place of the double layer capacitance (CDL). Note both circuits 46 and 44 contain an infinite Warburg element (ZW) in series with the charge transfer resistance (RCT).

As mentioned above, experiments were conducted to demonstrate ePEMC properties. Three experiments included: (1) resonant frequency response to electrochemically-deposited metal thin-films, (2) resonant frequency response to adsorption of thiolated ssDNA and model proteins with subsequent EIS sensing, and (3) simultaneous resonant frequency and charge transfer resistance response to model chemisorption of a short-chain thiol molecule, mercaptohexanol. Reagents utilized to conduct the aforementioned experiments included Concentrated sulfuric acid (H₂SO₄), 30% hydrogen peroxide (H₂O₂), sodium chloride (NaCl) and potassium chloride (KCl) were from Fisher Scientific. Thiolated DNA strand (HS-C₆T₆CCCTGAGTGTCAGATACAGCCCAGTAG) was purchased from Integrated DNA Technologies (IDT, Coralville, Iowa). Ethylenediaminetetraacetic acid (EDTA) and tris-hydrochloride (Tris-HCl) were from Sigma-Aldrich. DNA was reconstituted in Tris-EDTA buffer (10 mM Tris, 1 mM EDTA, pH=7.9, 1 M NaCl) and stored at −22° C. until removed for use. Ethanol (EtOH, 200 proof) was from Decon Laboratories, Inc (King of Prussia, Pa.). Bond-Breaker TCEP (tris(2-carboxyethyl)phosphine) solution (500 mM) used for reduction of disulfide form of DNA strands was from Fisher. Phosphate buffered saline (PBS, 10 mM, pH 7.4), potassium ferrocyanide (K₄Fe(CN)₆), potassium ferricyanide (K₃Fe(CN)₆), bovine serum albumin (BSA), and copper (II) sulfate (CuSO₄) were from Sigma-Aldrich. Polyurethane (MC, clear) was from Wassar Corporation (Auburn, Wash.). Lead zirconate titanate (PZT-5A) was from Piezo Systems (Woburn, Mass.). Deionized water (DIW, 18 MΩ, Milli-Q system, Millipore) was used for buffer preparation and rinsing protocols. Hydrogen tetrachloroaurate (III) trihydrate (HAuCl₄.H₂O) was purchased from Acros Organics. Adhesive copper tape for connection to potentiostat leads was from 3M.

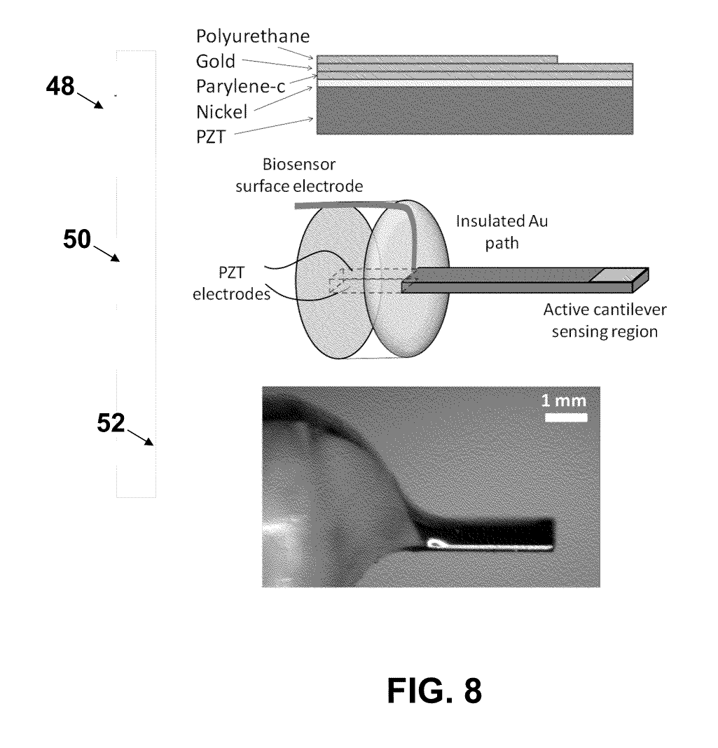

FIG. 8 illustrates diagrams 48 and 50, and photograph 52 of the ePEMC sensor utilized in the experiments. Diagram 48 depicts a two-dimensional 2D schematic of ePEMC cross section showing sensor piezoelectric-polymer-metal composite layers. Diagram 50 depicts three-dimensional (3D) schematic of the ePEMC design showing sensing electrode area at the cantilever distal tip and electrically-insulated conductive path for instrument interface. Photograph 52 is a photograph of the ePEMC sensor. To fabricate sensors, parylene-c coated sensors were fabricated from PZT-5A. A custom shadow mask was designed for depositing a 100 nm thick gold (Au) layer on the cantilever top surface and a conductive path. Adhesive copper tape was attached to the Au pathway near the cantilever base for interfacing with measurement instrument. The gold connection line was electrically-insulated by a spin-coated polyurethane layer (˜30 seconds at 1500 rpm) leaving only ˜1 mm² Au at the distal end for biochemical sensing.

To prepare the sensor surface, prior to conducting the biosensing experiments, the freshly sputtered 1 mm² Au electrode was cleaned in room temperature piranha solution (3:1 v/v H₂SO₄:H₂O₂) for ˜30 seconds. Caution: Piranha solution is highly reactive; handle with care. The sensor was then rinsed immediately with copious DIW and installed in the flow cell.

To prepare DNA, thiolated DNA was supplied by a vendor in disulfide form. The disulfide bond was reduced by adding 1 μL 500 mM TCEP to 300 μL of 1.4 μM DNA which was subsequently mixed and allowed to react ˜60 minutes at room temperature. The reduced-DNA was added to the re-circulating buffer (flow rate=500 μL/min) for chemisorbing DNA strand on the Au surface. Flow was maintained by a peristaltic pump.

Resonant frequency of the ePEMC was obtained by monitoring the PZT layer impedance-based frequency response at 100 mV sinusoidal driving input with zero bias using an impedance analyzer (Agilent Model 4294A). Sensor spectra were characterized by frequency sweep over the range of 1-250 kHz. Resonant frequency was determined from continual sweeping of frequency within 5-10 kHz of resonant frequency by a custom LabView® program. Resonance was identified by the frequency at maximum phase angle between the exciting voltage and the resulting current through the PZT.

The Au-layer on the ePEMC served as the working electrode. Copper sulfate (CuSO₄, 1 M, DIW) and hydrogen tetrachloroaurate (III) trihydrate salt (HAuCl₄.3H₂O, 50 mM, DIW) were used in the respective copper and gold half-cells. A salt bridge (saturated KCl, diameter ˜3 mm) was used to connect the two half cells. Voltage and current were measured by a multimeter (Fluka Model 289).

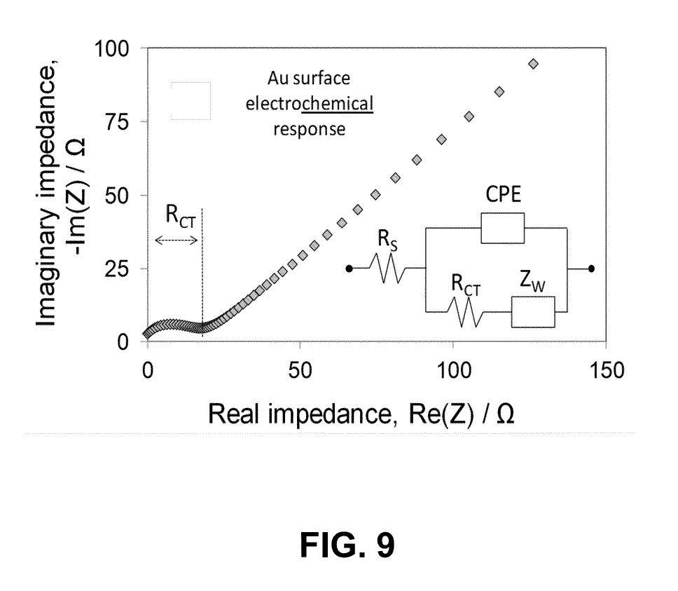

FIG. 9 is a plot of imaginary impedance versus real impedance obtained by a fit of data to a modified Randles equivalent circuit model. FIG. 9 depicts the EIS spectrum of the sensor measured in PBS buffered Fe(CN)64-/3- when ePEMC was in the second resonant mode. The inset circuit schematic diagram shows the best-fit modified Randles equivalent circuit containing solution resistance (RS), charge transfer resistance (RCT), constant phase element (CPE), and infinite Warburg element (ZW). Electromechanical impedance spectroscopy was achieved via a three-electrode arrangement using an ePEMC as the working electrode, silver/silver chloride (Ag/AgCl) as the reference electrode, and platinum wire (Pt) as the counter electrode. Electrochemical impedance spectra (EIS, Interface 1000 Gamry Instruments, Warminster, Pa.) were made in 50 mM Fe(CN)₆^{4-/3-} in PBS over the frequency range 10 mHz-100 kHz (DC bias=0 V) using a driving voltage of 10 mV with respect to the open circuit voltage. EIS spectra were normalized by shifting real part of the impedance, Re(Z), to the origin for comparison. R_{CT} was obtained by a fit of data to a modified Randles equivalent circuit model shown in FIG. 9. R_{CT} calculated from equivalent circuit models and from the x-axis semicircle distance formed by impedance data in the Nyquist plot agreed well within ˜6%. A comparison of equivalent circuit models is shown in FIG. 7.

To describe the electromechanical characterization of the results, resonant frequency of the n^{th} transverse mode depends on effective cantilever mass (m_{c}) and spring constant (k) as:

$$\text{?} = \text{?} \sqrt{\frac{\text{?}}{\text{?}}} \\ \text{?} \text{indicates text missing or illegible when filed} \qquad ( 1 )$$

where k_{eff}=Ewt³/(12L³), m_{c}=ρLwt, cₙ=λₙ²/2π, L, w, and t are the cantilever length, width, and thickness, respectively, ρ is the density, and λₙ is the corresponding eigenvalue. Thus, changes in frequency are associated with changes in both mass and stiffness as:

$$Δ f = \frac{1}{2} \text{?} ( \frac{Δ k}{k} - \frac{Δ m}{\text{?}} ) \\ \text{?} \text{indicates text missing or illegible when filed} \qquad ( 2 )$$

which reduces to the following when stiffness changes are negligible, as is the case for biomolecular surface reactions:

$$Δ f = - \frac{1}{2} \text{?} ( \frac{\text{?}}{\text{?}} ) \\ \text{?} \text{indicates text missing or illegible when filed} \qquad ( 3 )$$

From the above, it is evident that when mass of the sensor increases there is a corresponding decrease in resonant frequency.

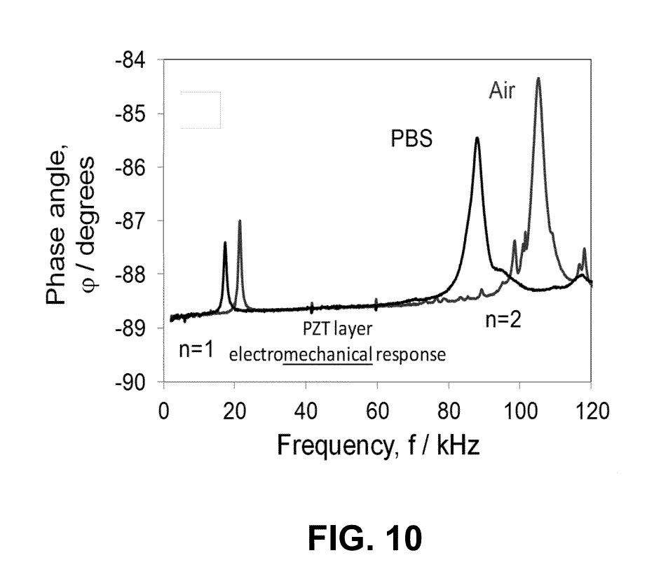

FIG. 10 illustrates example resonant mode graphs of phase angle versus frequency. FIG. 10 depicts the electromechanical resonance spectrum of ePEMC showing the first and second bending resonant modes. The resonant frequencies decrease when immersed in PBS due to added mass effect. EPEMC sensors showed two resonant modes at 21 and 105 kHz as shown by the electromechanical impedance response of the PZT layer over 0-120 kHz as shown in FIG. 10. It should be noted that the position of the two modes may be manipulated by suitable choice of sensor geometry and material as described in Equation 1. Upon immersion in a liquid, the two transverse modes decreased by 4.2±0.1 and 18.5±0.1 kHz, respectively. Since the cantilever Reynolds number was high (˜10⁴), the added mass effect of the surrounding liquid may have contributed to the resonant frequency shift.

Electrochemical impedance behavior measured using planar electrode biosensors may follow Randles or modified Randles equivalent circuit models. Similar to monitoring Δf, measurement of electrochemical impedance spectra facilitates monitoring changes in the charge transfer resistance (R_{CT}). As shown schematically in FIG. 5, R_{CT} also may be sensitive to immobilized biologics, and thus, monitoring changes in R_{CT} may be used in electrochemical biosensing applications for transducing binding of biomolecules. The ePEMC electrochemical impedance spectra (EIS) exhibited Randles-like behavior over 10 mHz-100 kHz as illustrated in FIG. 7 and FIG. 9. In the absence of bound biomolecules, the ePEMC Au surface exhibited R_{CT} of 22±8Ω (n=5 sensors).

The response of the ePEMC to spontaneous deposition of Au thin-films is now discussed. One way to demonstrate simultaneous chemical sensing is the application of the ePEMC to monitoring electrochemical metal thin-film deposition, since such reactions produce both an added mass effect and demonstrate the ability of the sensing surface to conduct electric current. The reduction of metal ions occurring on the gold surface of ePEMC is given by:

Mⁿ⁺+ne⁻→M(s),

where Mⁿ⁺ is the metal ion reduced on the sensor, e⁻ is an electron, n is the number of electrons, and M(s) is the solid metal deposited on ePEMC. Cu²⁺+2e⁻ was utilized as the counter half-cell to ensure spontaneous metal deposition on the sensor surface given its standard reduction potential is less positive than that of most gold cations and cation-complexes.

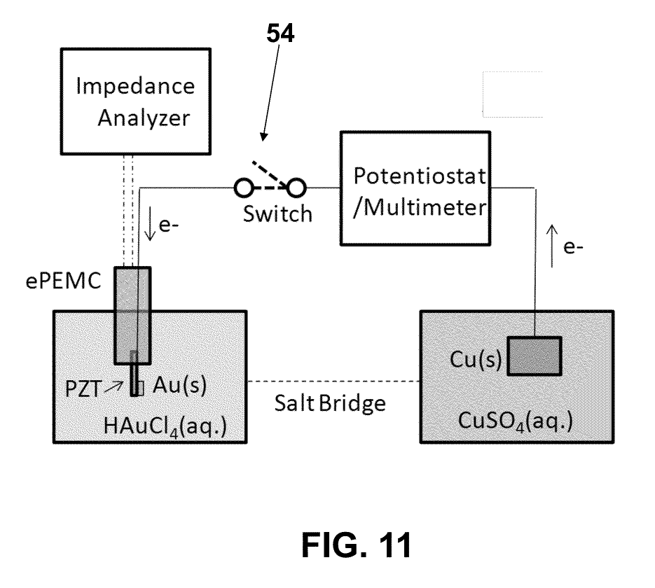

FIG. 11 is a schematic diagram of an example circuit utilized to depose gold on the surface of the sensor. FIG. 11 depicts a schematic of the Au thin-film deposition configuration. The anode is solid copper and the cathode is the Au-surface on ePEMC. A salt bridge provided an electrical path between the two half-cells. An electrical switch for closing and breaking the circuit facilitates cycles of deposition. The deposition of Au on the sensor can be controlled by the switch 54 in the circuit shown in FIG. 11.

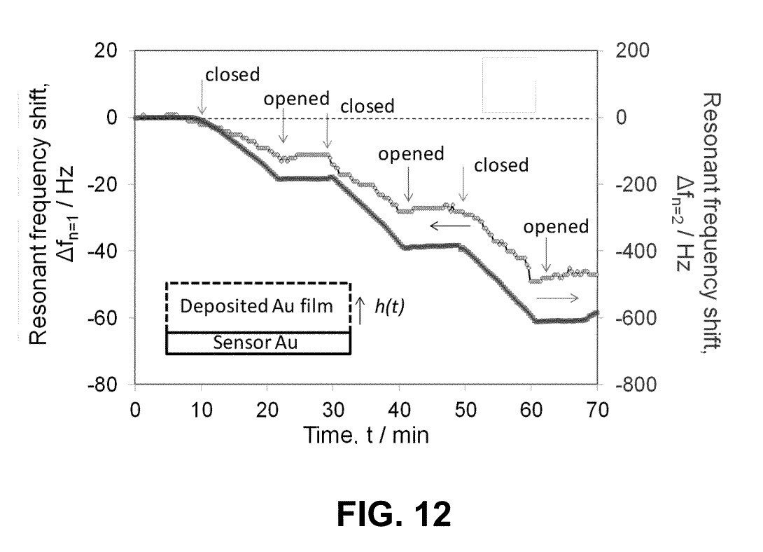

FIG. 12 illustrates the response of the first and second bending modes and voltage during gold deposition cycles on the ePEMC sensors. As shown in FIG. 12, the ePEMC resonant frequency was first allowed to stabilize in the configuration shown with the switch 54 in the open position which prevented gold deposition. After a steady-state in resonant frequency was established, the switch 54 was closed which allowed the spontaneous reduction reaction to occur on ePEMC gold surface. Both the resonant frequency and cell potential were recorded continuously. As shown in FIG. 12, closing the switch 54 caused a linear resonant frequency decrease at a rate of 1.4±0.5 and 16.4, 4.6 Hz/min for the first and second modes, respectively. The measured cell potential was 0.8 V during the switch-closed periods which suggests a spontaneous one electron reduction of [AuCl₂]⁻ on the sensor Au surface according to: [AuCl₂]⁻+e⁻→Au(s)+2Cl⁻ and a net cell reaction: 2[AuCl₂]⁻+Cu(s)→2Au(s)+Cu²⁺+4Cl⁻. The measured potential agreed reasonably with the expected value (E°_{cell}) calculated using standard reduction potentials)(E°) (E°_{cell}=E°_{cathode}−E°_{anode}=(+1.15 V)−(+0.34 V)=0.81 V). Absence of three electron reduction potential may be due to oxidation of the tetrachloroaurate ion, [AuCl₄]⁻, to [AuCl₂]⁻, which occurs readily in aqueous solution in presence of light and heat. After Au thin-film deposition occurred for ˜10 minutes, the switch 54 was opened which caused the resonant frequency to stabilize and the measured cell potential to decrease to 0 V. FIG. 12 shows that the sensor response was repeatable when subjected to successive cycles of Au deposition on the sensor surface. These results may indicate that the ePEMC simultaneously measures mass-change and electric current through the Au sensing surface.

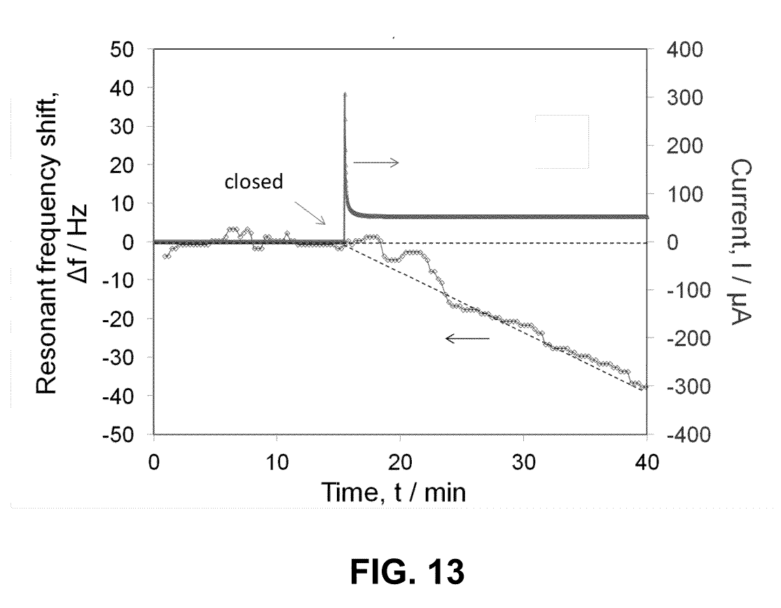

FIG. 13 illustrates current and resonant frequency transients during one cycle. For time less than 15 minutes, the circuit switch was open and the resonant frequency remained constant. Upon closing the switch, the current increased from 0 to ˜50 μA, and the resonant frequency began to decrease at a constant rate due to gold deposition on the ePEMC. As shown in FIG. 13, the current during the deposition period was ˜50 μA. Since one mole of e⁻ is generated per mole of [AuCl₂]⁻ reduced, the Au deposition rate may be estimated as: (53 μA)/(96,485 C/mol e⁻)×(1 mol Au(s)/1 mol e⁻)=550 picomoles Au(s)/s. Therefore, estimated mass deposition rate was 110 ng/s. Since the rate of resonant frequency decrease was 1.7 Hz/min during deposition, the mass-change sensitivity to mass deposited in the form of a thin-film was 3.6 mg/Hz. Interestingly, this value is significantly lower than resonant frequency decrease observed for added mass due to molecular binding which does not form a coherent film. This may be attributed to the reduction in observed mass change sensitivity to the metal film formation which has a potential stiffness contribution, and lowers mass change sensitivity, as given by Equation (2). The ePEMC responses to surface molecular binding reactions were examined using both a model protein and thiolated single-stranded DNA (ssDNA).

Bovine serum albumin (BSA) and linear molecules containing thiol end groups were chosen to examine the ePEMC's potential in biosensing applications for surface-based biosensing because they readily bind to Au. A typical binding experiment, started with cleaning the gold surface, followed by measuring the sensor's electrochemical impedance spectrum. Subsequently, the sensor was installed in a flow cell for mass-change sensing, thus allowing the sensor to stabilize in flowing PBS at 500 μL/min.

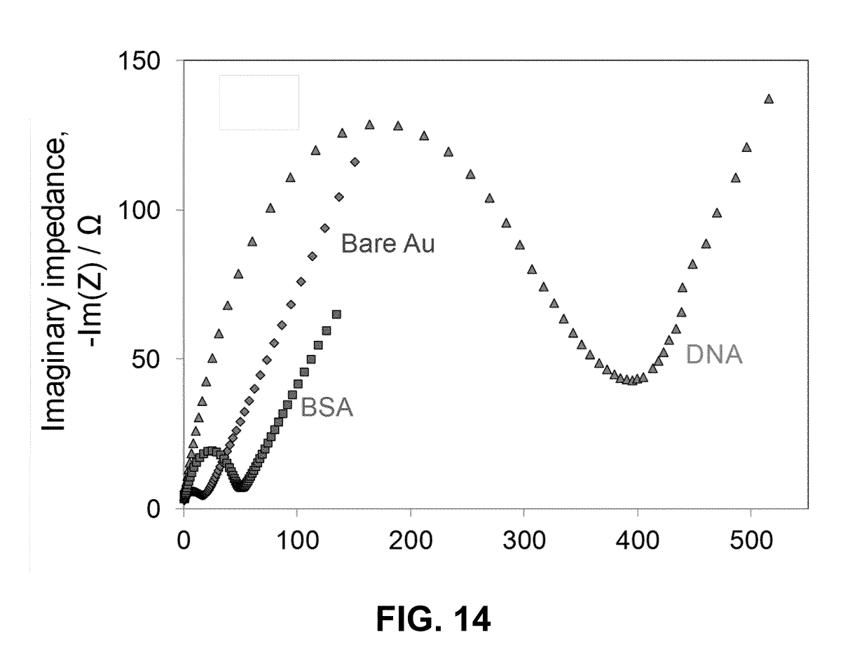

FIG. 14 illustrates the EIS spectrum of ePEMC to protein and thiolated ssDNA. As shown in FIG. 14, a freshly cleaned ePEMC gave R_{CT} of 22±8Ω (n=5 different sensors). After a constant baseline in resonant frequency was obtained, the flow was switched from buffer to either DNA (1.4 nM) or BSA (200 μg/mL) solutions and the flow was put in a re-circulation mode until binding steady state was reached.

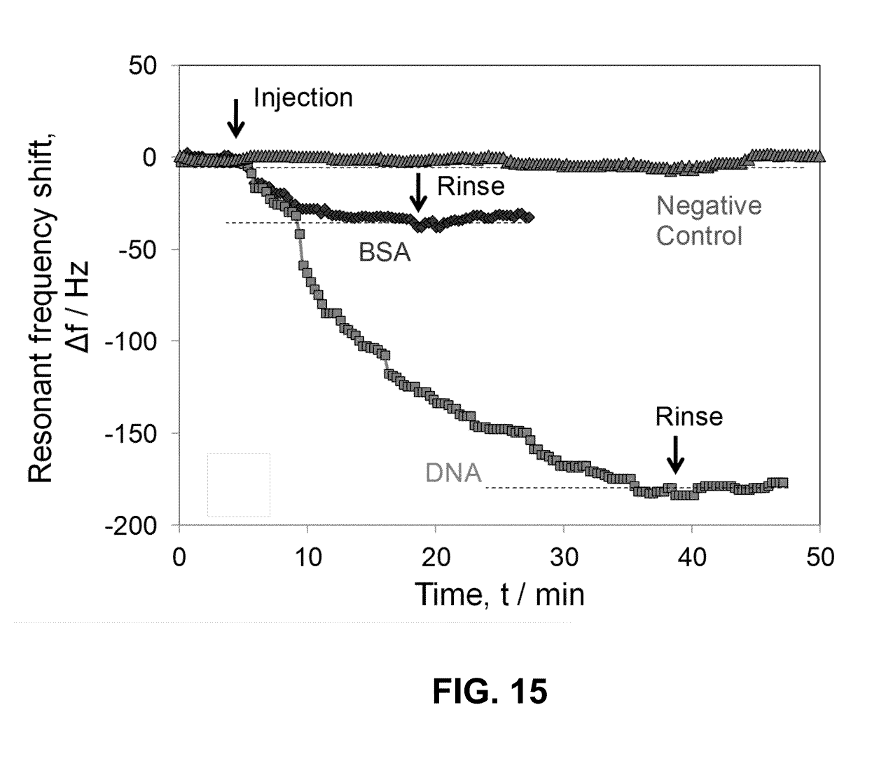

FIG. 15 illustrates the electromechanical response for the same adsorption. Negative control is the response to injection of a solution absent of binding analyte. FIG. 15 illustrates example plots of resonant frequency shift versus time. As shown in FIG. 15, adsorption of BSA typically occurred in 15 minutes causing a mass-change response of 30±7 Hz shift (n=2) in resonant frequency. The sensor was rinsed in situ by returning the flow to PBS which did not cause any resonant frequency change, indicating that the previous shift was caused by protein adsorption and was not due to any other effects. The sensor was then removed and the electrochemical impedance spectrum was re-measured. The sensor showed R_{CT} increased to 50±18Ω (n=2) after BSA was adsorbed, as shown in FIG. 14.

In the second experiment, chemisorption of thiolated ssDNA took ˜40 minutes causing a 180±22 Hz shift (n=2) in resonant frequency as shown in FIG. 15. Similar to the case of the BSA experiments, in situ rinse with TE buffer caused no shift in resonant frequency indicating the response was due to chemisorption of the ssDNA. As shown in FIG. 14, the sensor also showed significant increase in R_{CT} (394±65Ω; n=2) which was found to be larger than for BSA, suggesting a more dense surface coverage. These results suggest that the ePEMC gives the ability to monitor surface-based biomolecular reactions by the two transduction mechanisms, Δf and ΔR_{CT}. It should be noted that increase of R_{CT} accompanying thiolated nucleic acid hybridization has provided femtomolar level sensitivity in nucleic acid biosensing applications. This is similar to the mass-based sensitivity for PEMC biosensors to nucleic acid hybridization which is the electromechanical component of the ePEMC device.

The results of simultaneous sensing of molecular self-assembly via changes in resonant frequency and charge transfer resistance are now described. The potential for making both measurements simultaneously during the course of surface molecular binding is examined Thus, sensor response to chemisorption of short chain (C₆) thiol molecule, mercaptohexanol (MCH), in the absence of flow while tracking both change in resonant frequency and charge transfer resistance. was examined

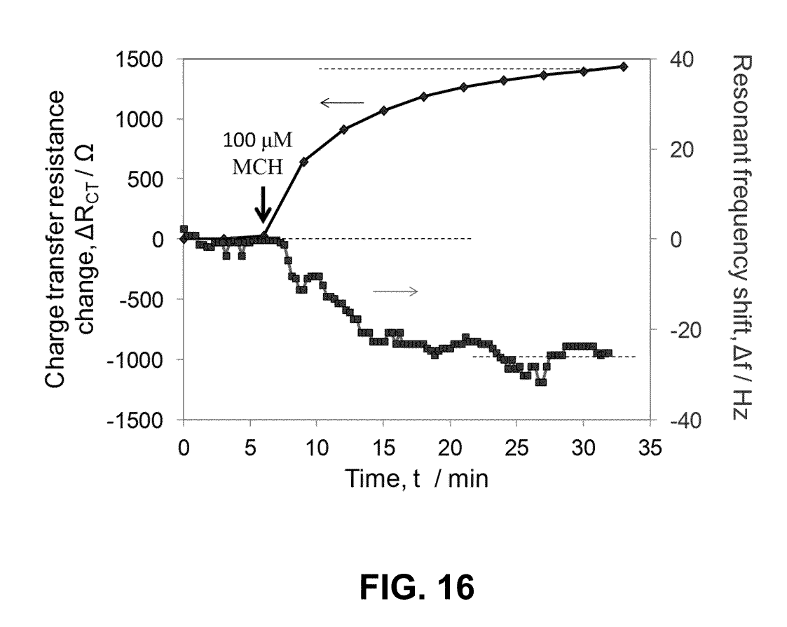

FIG. 16 illustrates simultaneously measured resonant frequency and charge transfer resistance responses of the ePEMC sensor to MCH chemisorption in absence of flow. FIG. 16 illustrates plots of charge transfer resistance changes versus time. As shown in FIG. 16, in the absence of any binding analyte, both resonant frequency and charge transfer resistance remain constant. However, upon introduction of MCH (100 μL at 100 μM) into the solution, resonant frequency decreased and charge transfer resistance increased in an exponential manner and took ˜15 to 25 minutes to reach steady state. Binding of MCH caused a decrease in resonant frequency (Δf˜30 Hz) and an increase in charge transfer resistance (ΔR_{CT}˜1.5 kΩ). The shift in resonant frequency compared to earlier response to ssDNA chemisorption was due to the use of the sensor post-BSA incubation and lower relative molecular weight of MCH compared to DNA (MW˜7 kDa). As shown in FIG. 16, resonant frequency response reached steady state value by t=20 minutes, while ΔR_{CT} is yet to reach steady state at 32 minutes. Since ΔR_{CT} transient depends on how densely MCH is assembled on the electrode surface, it may be that the response following t=20 minutes is a rearrangement on the Au surface. The resonant frequency response does not distinguish the actual arrangement on the sensor, but simply the attachment.

Sensor resonant frequency decreases for BSA and MCH, shown in FIG. 15 and in FIG. 16, suggest approximately the same mass of BSA and MCH became bound to the sensor. Comparison of the corresponding electrochemical responses suggests that the MCH induced a far greater charge transfer resistance than BSA. This difference is attributed to the dense surface density of MCH due to self-assembled monolayers on sensor's Au<111> sites. Thus, the ePEMC-based measurement provides two complementary sensitive assays, namely changes in mass and changes in charge density. Since the measurements are obtained simultaneously and dynamically, additional insights on surface binding phenomena can be obtained, which are not as easily obtained by a single transduction mechanism.

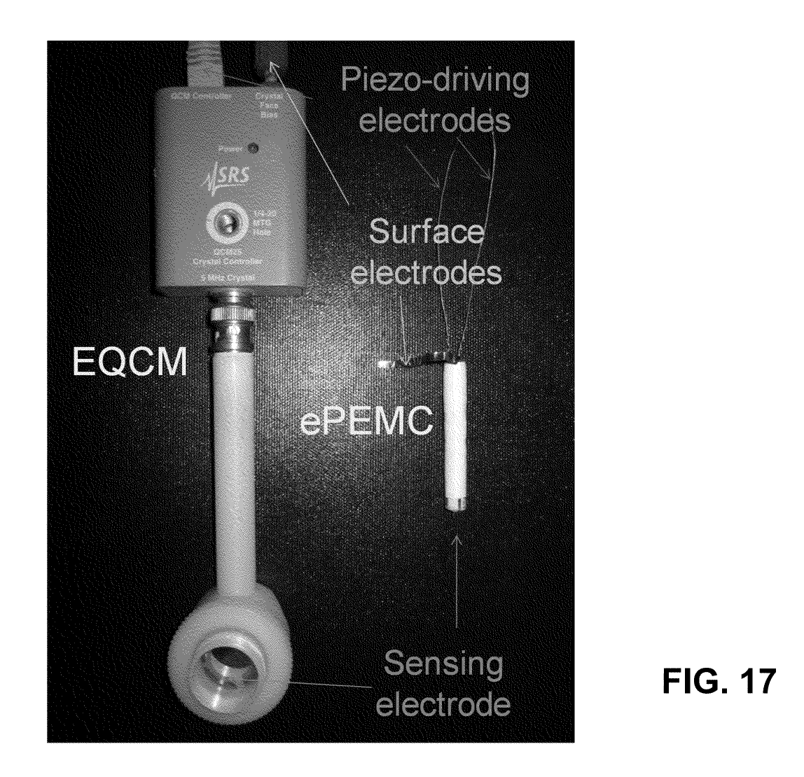

FIG. 17 illustrates a pictorial comparison of the ePEMC sensor with a commercially available electrochemical-quartz crystal microbalance (EQCM) sensor.

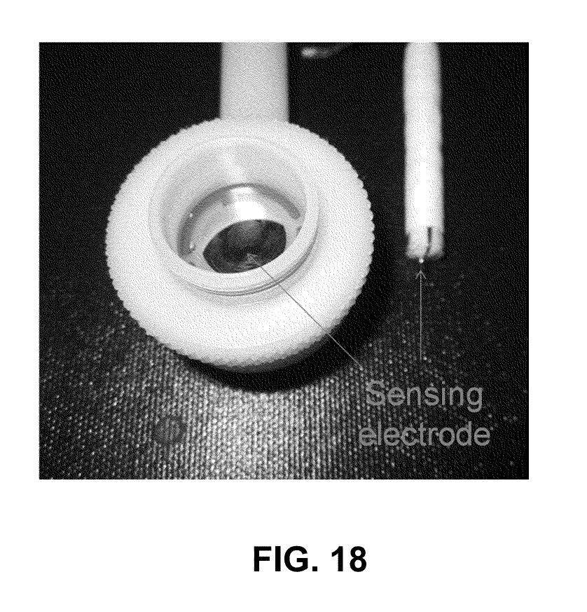

FIG. 18 is a pictorial view of the sensor working electrodes. The EQCM sensor has a working electrode area on the order of ˜cm², and the ePEMC sensor has a working electrode area on the order of ˜mm².

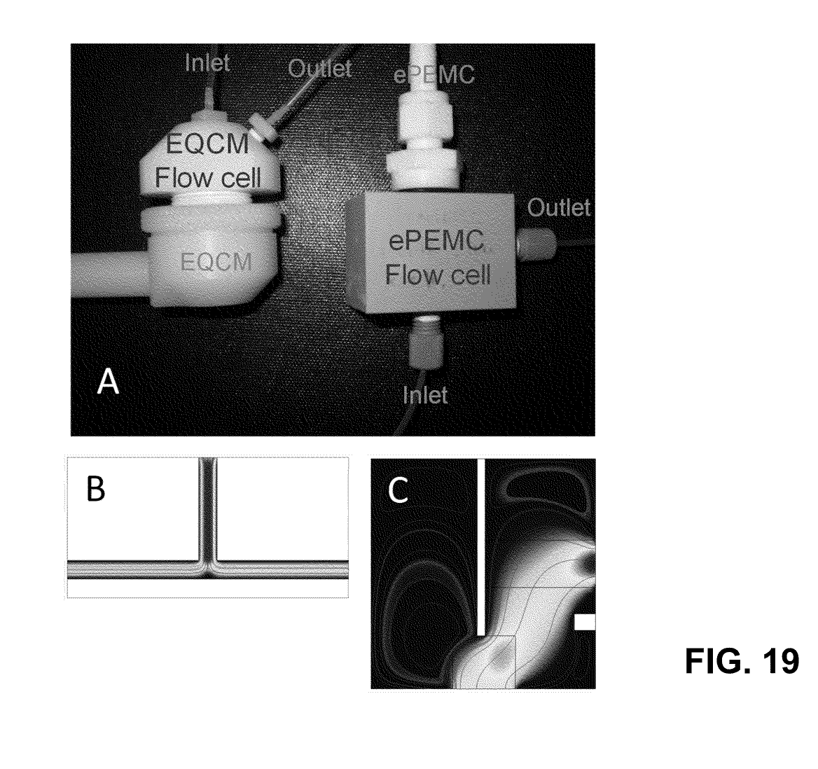

FIG. 19, diagram A is a pictorial illustration of the flow cells used for EQCM sensing and ePEMC sensing. Reactor hold-up volumes were 150 μL and ˜300 μL, respectively. Finite element modeling software (COMSOL Multiphysics, Vers. 3.5a) was used to calculate flow cell velocity profiles in both the commercially available EQCM, as depicted in diagram B, and the ePEMC device, as depicted in diagram C. Solution was examined in 2-dimensions (2D) using Lagrange-P2P1 type elements.

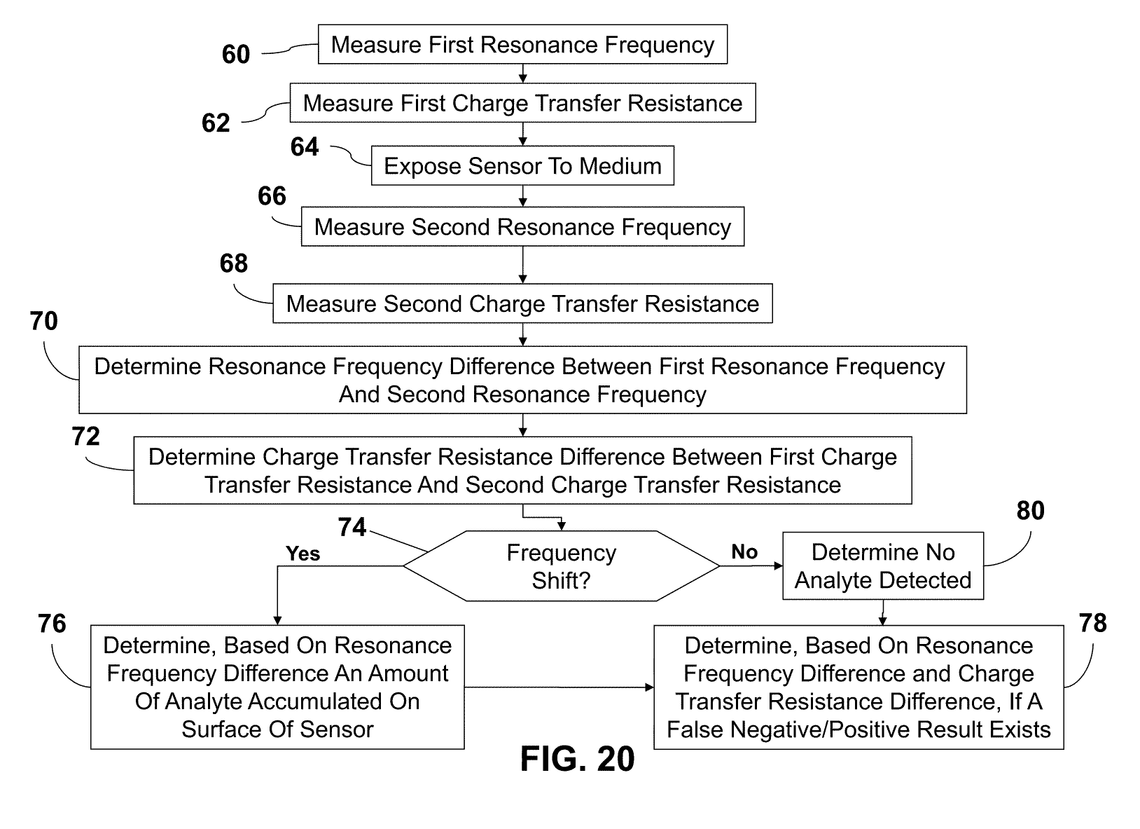

FIG. 20 is a flow diagram of an example process for facilitating dual mode sensing on a single sensor. The sensor can be configured in accordance with the descriptions provided herein, or configured in accordance with any appropriate variant thereof. A first resonance frequency of the sensor may be measured at step 60. The first resonance frequency may be measured as described herein and/or in accordance with any appropriate variant thereof. The resonance frequency may be measured by any appropriate means, such as an operational amplifier, an impedance analyzer, a network analyzer, an oscillator circuit, or the like, for example. When the piezoelectric material of the piezoelectric portion of the sensor is excited, the non-piezoelectric portion of the sensor may flex to accommodate the strain caused in the piezoelectric material. When the frequency of excitation is the same as the natural frequency of the underlying mechanical structure, resonance occurs.

A first charge transfer resistance of the sensor may be measured at step 62. The first charge transfer resistance may be measured as described herein and/or in accordance with any appropriate variant thereof.

The sensor may be prepared to receive an analyte. In an example embodiment, an analyte attractor is applied to the sensor. The attractor is specific to a target analyte. Thus the attractor will attract a target analyte and not attract other substances. For example, the sensor may comprise an attractor for attracting Bacillus anthracis, food-borne pathogens, such as E. coli, pathogens in food and water, cell types in body fluids (e.g., circulating tumor cells), biomarkers in body fluids (e.g., proteins that mark specific pathophysiology—alpha-fetoprotein, beta-2-microglobulin, bladder tumor antigen, breast cancer marker CA-15-3, and other CAs (cancer antigens), calcitonin, carcinoembryonic antigen, and others), markers of explosives such as trinitrotoluene, dinitrotoluene, airborne and waterborne toxins, biological entities, such as a protein, DNA, or any appropriate combination thereof.

The sensor may be exposed to a medium at step 64. The medium may comprise any appropriate medium, such as a liquid, a gas, a combination of a liquid and a gas, or a vacuum, for example. The medium may exhibit a wide variety of flow conditions. If a target analyte is present in the medium, the target analyte may accumulate on the surface of the sensor that has been treated with the attractor. As described herein, accumulation (e.g., binding) of the target analyte on the surface of the sensor may result in a change in stiffness of the sensor and/or an increase the mass of the sensor, which will decrease the resonance frequency of the sensor.

A second resonance frequency of the sensor may be measured at step 66. The second resonance frequency may be measured as described herein and/or in accordance with any appropriate variant thereof. A second charge transfer resistance of the sensor may be measured at step 68. The second charge transfer resistance may be measured as described herein and/or in accordance with any appropriate variant thereof.

The second measured resonance frequency may be compared to a baseline resonance frequency. That is, a difference between the first resonance frequency (baseline) and the second resonance frequency may be determined at step 70. The baseline resonance frequency may be the resonance frequency of the sensor having no analyte accumulated thereon. If a difference in the measured (first and second) resonance frequencies (frequency shift) is not measured (at step 74), it is determined, at step 80, that no analyte is detected. If a difference in frequency between the measured resonance frequency and the baseline resonance frequency is measured (at step 74), it is determined, at step 76, that an analyte is detected, i.e., an analyte is present in the medium. Additionally, based on the resonance frequency difference, an amount of analyte accumulated on the sensor may be determined at step 76.

The second charge transfer resistance measurement may be compared to a baseline charge transfer resistance. That is, a difference between the first charge transfer resistance (baseline) and the second resonance frequency may be determined at step 78. If the charge transfer resistance difference is below a threshold value, it may be determined that the determination that no analyte has accumulated on the surface of the sensor (step 80) is not a false negative result. If charge transfer resistance difference is above a threshold value, it may be determined that the determination that analyte has accumulated on the surface of the sensor (step 76) is not a false positive result.

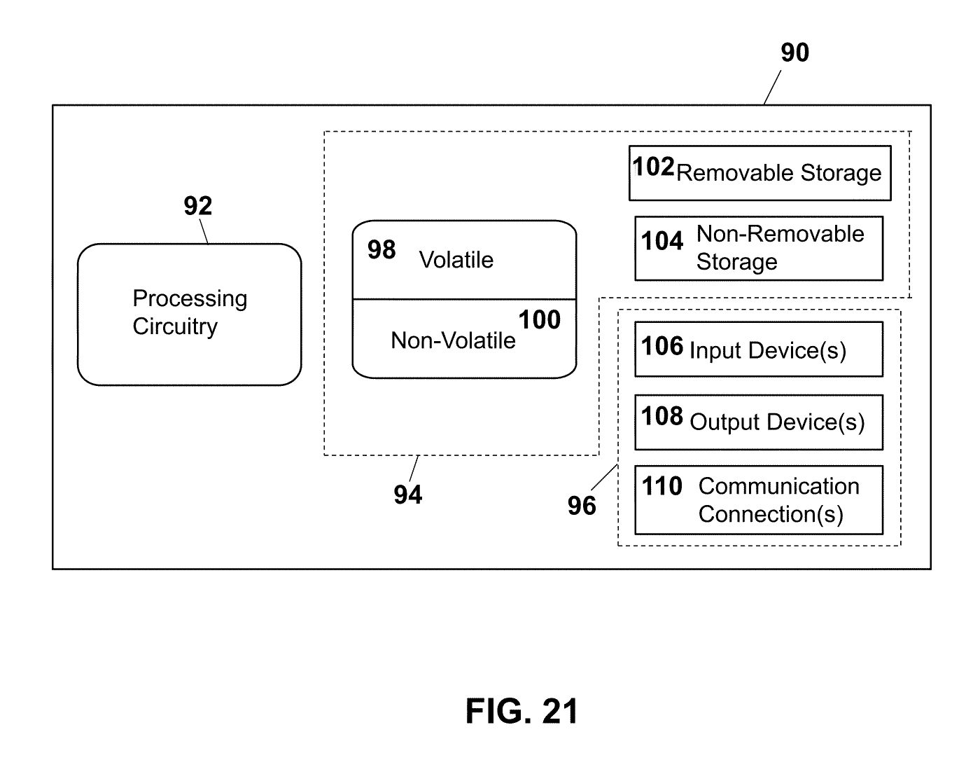

FIG. 21 is a block diagram of an example apparatus 90 that may be utilized to effectuate dual mode sensing on a single sensor as described herein. The apparatus 90 may comprise hardware or a combination of hardware and software. The apparatus 90 depicted in FIG. 21 may represent any appropriate, or appropriate combination of apparatuses. It is emphasized that the block diagram depicted in FIG. 21 is exemplary and not intended to imply a specific implementation or configuration. Thus, the apparatus 90 may be implemented in a single apparatus or multiple apparatuses (e.g., single server or multiple servers, single gateway or multiple gateways, single computer or multiple computers, single tablet device or multiple tablet device, etc.), or any appropriate combination thereof. Multiple apparatuses may be distributed or centrally located. Multiple apparatuses may communicate wirelessly, via hard wire, or any appropriate combination thereof.

In an example embodiment, the apparatus 90 may comprise a processor and memory coupled to the processor. The memory may comprise executable instructions that when executed by the processor cause the processor to effectuate operations associated with dual mode sensing as described herein. As evident from the herein description, apparatus 90 is not to be construed as software per se.

In an example configuration, the apparatus 90 may comprise a processing portion 92, a memory portion 94, and an input/output portion 96. The processing portion 92, memory portion 94, and input/output portion 96 may be coupled together (coupling not shown in FIG. 3) to allow communications therebetween. Each portion of the apparatus 90 may comprise circuitry for performing functions associated with each respective portion. Thus, each portion may comprise hardware, or a combination of hardware and software. Accordingly, each portion of the apparatus 90 is not to be construed as software per se. The input/output portion 96 may be capable of receiving and/or providing information from/to a communications device and/or other apparatuses configured for effectuating dual mode sensing as described herein. For example, the input/output portion 96 may include a wireless communications (e.g., 2.5G/3G/4G/5G/GPS) card. The input/output portion 96 may be capable of receiving and/or sending video information, audio information, control information, image information, data, or any combination thereof. In an example embodiment, the input/output portion 96 may be capable of receiving and/or sending information to determine a location of the apparatus 90 and/or the communications apparatus 90. In an example configuration, the input\output portion 96 may comprise a GPS receiver. In an example configuration, the apparatus 90 may determine its own geographical location and/or the geographical location of a communications device through any type of location determination system including, for example, the Global Positioning System (GPS), assisted GPS (A-GPS), time difference of arrival calculations, configured constant location (in the case of non-moving devices), any combination thereof, or any other appropriate means. In various configurations, the input/output portion 96 may receive and/or provide information via any appropriate means, such as, for example, optical means (e.g., infrared), electromagnetic means (e.g., RF, WI-FI, BLUETOOTH, ZIGBEE, etc.), acoustic means (e.g., speaker, microphone, ultrasonic receiver, ultrasonic transmitter), or a combination thereof. In an example configuration, the input/output portion may comprise a WIFI finder, a two way GPS chipset or equivalent, or the like, or a combination thereof.

The processing portion 92 may be capable of performing functions associated with dual mode sensing as described herein. For example, the processing portion 92 may be capable of, in conjunction with any other portion of the apparatus 90, installing an application for effectuating dual mode sensing as described herein.

In a basic configuration, the apparatus 90 may include at least one memory portion 94. The memory portion 94 may comprise a storage medium having a concrete, tangible, physical structure. Thus, the memory portion 94, as well as any computer-readable storage medium described herein, is not to be construed as a transient signal per se. The memory portion 94, as well as any computer-readable storage medium described herein, is not to be construed as a propagating signal per se. The memory portion 94, as well as any computer-readable storage medium described herein, is not to be construed as a signal per se. The memory portion 94, as well as any computer-readable storage medium described herein, is to be construed as an article of manufacture. The memory portion 94 may store any information utilized in conjunction with effectuating dual mode sensing as described herein. Depending upon the exact configuration and type of processor, the memory portion 94 may be volatile 98 (such as some types of RAM), non-volatile 100 (such as ROM, flash memory, etc.), or a combination thereof. The apparatus 90 may include additional storage (e.g., removable storage 102 and/or non-removable storage 104) including, for example, tape, flash memory, smart cards, CD-ROM, digital versatile disks (DVD) or other optical storage, magnetic cassettes, magnetic tape, magnetic disk storage or other magnetic storage devices, universal serial bus (USB) compatible memory, or any other medium which can be used to store information and which can be accessed by the apparatus 90.

The apparatus 90 also may contain communications connection(s) 110 that allow the apparatus 90 to communicate with other devices, network entities, or the like. A communications connection(s) may comprise communication media. Communication media may embody computer readable instructions, data structures, program modules or other data in a modulated data signal such as a carrier wave or other transport mechanism and includes any information delivery media. By way of example, and not limitation, communication media may include wired media such as a wired network or direct-wired connection, and wireless media such as acoustic, RF, infrared, and other wireless media. The term computer readable media as used herein includes both storage media and communication media. The apparatus 90 also may include input device(s) 106 such as keyboard, mouse, pen, voice input device, touch input device, etc. Output device(s) 108 such as a display, speakers, printer, etc. also may be included.

As described herein, a new dual mode sensing platform capable of robust biochemical sensing comprises the integration, in a piezoelectric cantilever sensor, of electrochemical sensing capabilities with mass sensing measurement capabilities. Validation of the ePEMC's electrochemical and mass sensing features were shown by examining a number of surface binding experiments, including metal thin-film deposition, protein adsorption, and chemisorption of thiolated molecules (short-chain thiols and ssDNA).

It is to be understood that even though numerous characteristics and advantages of dual mode sensors have been set forth in the foregoing description, together with details of the structure and function, the instant disclosure is illustrative only, and changes may be made in detail, especially in matters of shape, size, and arrangement of parts within the principles of asymmetric sensors to the full extent indicated by the broad general meaning of the terms in which the appended claims are expressed.

While example embodiments of dual mode sensors have been described in connection with various computing devices/processors, the underlying concepts may be applied to any computing device, processor, or system capable of dual mode measurement. The various techniques described herein may be implemented in connection with hardware or software or, where appropriate, with a combination of both. Thus, the methods and apparatuses associated with dual mode sensors, or certain aspects or portions thereof, may take the form of program code (i.e., instructions) embodied in tangible computer-readable storage media. Examples of tangible computer-readable storage media include floppy diskettes, CD-ROMs, DVDs, hard drives. When the program code is loaded into and executed by a machine, such as a computer, the machine becomes an apparatus for implementation of dual mode sensors. In the case of program code execution on programmable computers, the computing device will generally include a processor, a storage medium readable by the processor (including volatile and non-volatile memory and/or storage elements), at least one input device, and at least one output device. The program(s) can be implemented in assembly or machine language, if desired. The language can be a compiled or interpreted language, and combined with hardware implementations. As evident from the herein description, a tangible computer-readable storage medium is not to be construed as a signal. As evident from the herein description, a tangible computer-readable storage medium is not to be construed as a propagating signal. As evident from the herein description, a tangible computer-readable storage medium is not to be construed as a transient signal. As evident from the herein description, a tangible computer-readable storage medium is an article of manufacture.

The methods and apparatuses associated with dual mode sensors also may be practiced via communications embodied in the form of program code that is transmitted over some transmission medium, such as over electrical wiring or cabling, through fiber optics, or via any other form of transmission, wherein, when the program code is received and loaded into and executed by a machine, such as an EPROM, a gate array, a programmable logic device (PLD), a client computer, or the like, the machine becomes an apparatus for detection and measurement of mass change using impedance determinations. When implemented on a general-purpose processor, the program code combines with the processor to provide a unique apparatus that operates to effectuate processes associated with asymmetric sensors.

What is claimed:

1. A method comprising:
performing a mass-sensing measurements via a sensor;
performing a impedance measurements via the sensor; and
determining, based on the mass-sensing measurements and the impedance measurements, whether an analyte has accumulated on the sensor.

2. The method of claim 1, further comprising:
determining, based on the mass-sensing measurements and the impedance measurements, an amount of target analyte accumulated on the sensor.

3. The method of claim 1, further comprising:
performing a first mass-sensing measurement prior to exposing the sensor to a medium;
performing a second mass-sensing measurement while the sensor is exposed to the medium; and
determining, based on a difference between the first mass-sensing measurement and the second mass-sensing measurement, whether the analyte has accumulated on the sensor.

4. The method of claim 1, further comprising:
performing a first impedance measurement prior to exposing the sensor to a medium; and
performing a second impedance measurement while the sensor is exposed to the medium; and
determining, based on a difference between the first impedance measurement and the second impedance measurement, whether the analyte has accumulated on the sensor.

5. The method of claim 1, wherein:
the mass-sensing measurements comprise resonance frequency measurements.

6. The method of claim 1, wherein:
the impedance measurements comprise charge transfer resistance measurements.

7. The method of claim 1, further comprising:
determining, based on the mass-sensing measurements and the impedance measurements, whether a false negative result exists.

8. An apparatus comprising:
a processor; and
memory coupled to the processor, the memory comprising executable instructions that when executed by the processor cause the processor to effectuate operations comprising:
performing a mass-sensing measurements via a sensor;
performing a impedance measurements via the sensor; and
determining, based on the mass-sensing measurements and the impedance measurements, whether an analyte has accumulated on the sensor.

9. The apparatus of claim 8, the operations further comprising:
determining, based on the mass-sensing measurements and the impedance measurements, an amount of target analyte accumulated on the sensor.

10. The apparatus of claim 8, operations further comprising:
performing a first mass-sensing measurement prior to exposing the sensor to a medium;
performing a second mass-sensing measurement while the sensor is exposed to the medium; and
determining, based on a difference between the first mass-sensing measurement and the second mass-sensing measurement, whether the analyte has accumulated on the sensor.

11. The apparatus of claim 8, the operations further comprising:
performing a first impedance measurement prior to exposing the sensor to a medium; and
performing a second impedance measurement while the sensor is exposed to the medium; and
determining, based on a difference between the first impedance measurement and the second impedance measurement, whether the analyte has accumulated on the sensor.

12. The apparatus of claim 8, wherein:
the mass-sensing measurements comprise resonance frequency measurements.

13. The apparatus of claim 8, wherein:
the impedance measurements comprise charge transfer resistance measurements.

14. The apparatus of claim 8, the operations further comprising:
determining, based on the mass-sensing measurements and the impedance measurements, whether a false negative result exists.

15. A computer-readable storage medium comprising executable instructions that when executed by a processor cause the processor to effectuate operation comprising:
performing a mass-sensing measurements via a sensor;
performing a impedance measurements via the sensor; and
determining, based on the mass-sensing measurements and the impedance measurements, whether an analyte has accumulated on the sensor.

16. The computer-readable storage medium of claim 15, the operations further comprising:
determining, based on the mass-sensing measurements and the impedance measurements, an amount of target analyte accumulated on the sensor.

17. The computer-readable storage medium of claim 15, operations further comprising:
performing a first mass-sensing measurement prior to exposing the sensor to a medium;
performing a second mass-sensing measurement while the sensor is exposed to the medium;
performing a first impedance measurement prior to exposing the sensor to a medium;
performing a second impedance measurement while the sensor is exposed to the medium; and
determining, based on a difference between the first mass-sensing measurement and the second mass-sensing measurement and a difference between the first impedance measurement and the second impedance measurement, whether the analyte has accumulated on the sensor.

18. The computer-readable storage medium of claim 15, wherein:
the mass-sensing measurements comprise resonance frequency measurements.

19. The computer-readable storage medium of claim 15, wherein:
the impedance measurements comprise charge transfer resistance measurements.

20. The computer-readable storage medium of claim 15, the operations further comprising:
determining, based on the mass-sensing measurements and the impedance measurements, whether a false negative result exists.
